(19) United States
(12) Patent Application Publication
(10) Pub. No.: US 2026/0112457 A1
(43) Pub. Date: Apr. 23, 2026

(54) MACHINE LEARNING FRAMEWORK FOR PREDICTION AND ASSIGNMENT OF 2D NUCLEAR MAGNETIC RESONANCE (NMR) SPECTRA

(71) Applicant: Brandeis University, Waltham, MA (US)

(72) Inventors: Pengyu Hong, Wellesley, MA (US); Yunrui Li, Ashland, MA (US); Hao Xu, Waltham, MA (US)

(21) Appl. No.: 19/361,157

(22) Filed: Oct. 17, 2025

Related U.S. Application Data
(60) Provisional application No. 63/708,341, filed on Oct. 17, 2024.

(51) Int. Cl. G16C 20/20; G16C 20/70

(52) U.S. Cl. CPC G16C 20/20; G16C 20/70

(57) ABSTRACT

The present disclosure is related generally to a method including processing, by a model: a molecular graph associated with a molecular structure; and solvent information of a solvent associated with the molecular structure. The method includes predicting nuclear magnetic resonance (NMR) shifts associated with the molecular structure based on processing the molecular graph and the solvent information.

This application claims the benefit of U.S. Provisional Application No. 63/708,341 filed Oct. 17, 2024, the disclosure of which is incorporated herein by reference in its entirety.

### BACKGROUND

### Field

The present disclosure is related generally to nuclear magnetic resonance (NMR) spectroscopy, and more particularly, to a transductive machine learning framework for accurate prediction and assignment of 2D nuclear magnetic resonance (NMR) spectra. The present disclosure is related to solvent-aware 2D NMR prediction via multi-tasking pre-training and iterative unsupervised learning.

### Background Information

NMR spectroscopy is crucial across diverse scientific fields, revealing detailed structural information, electronic properties, and molecular dynamic insights. Accurate prediction of NMR peaks in a spectrum from molecular structures allows chemists to effectively evaluate candidate structures by comparing predictions with actual shifts in experimental NMR spectra. This process facilitates peak assignments, thereby aiding in verifying molecular structures or identifying discrepancies.

### SUMMARY

Embodiments of the invention are also directed to computer-implemented methods and computer program products having substantially the same features and functionality as a computer system described herein.

Embodiments of the present disclosure are directed to a method including: processing, by a model: a molecular graph associated with a molecular structure; and solvent information of a solvent associated with the molecular structure; and predicting nuclear magnetic resonance (NMR) shifts associated with the molecular structure based on processing the molecular graph and the solvent information.

In any one or combination of the embodiments disclosed herein, the NMR shifts include at least one of: hydrogen NMR shifts associated with the molecular structure; and carbon NMR shifts associated with the molecular structure.

In any one or combination of the embodiments disclosed herein, the method further includes: generating a concatenated representation associated with the molecular structure and the solvent, based on processing the molecular graph and the solvent information; and processing, by one or more multilayer perceptron (MLP) components included in the model, the concatenated representation, wherein predicting the NMR shifts is based on processing the concatenated representation.

In any one or combination of the embodiments disclosed herein, the one or more MLP components include: a first MLP component configured to predict carbon NMR shifts associated with the molecular structure; and a second MLP component configured to predict hydrogen NMR shifts associated with the molecular structure.

In any one or combination of the embodiments disclosed herein, the method further includes training the model based on annotated NMR data included in an annotated dataset.

In any one or combination of the embodiments disclosed herein, training the model includes multi-task pre-training of the model based on the annotated NMR data.

In any one or combination of the embodiments disclosed herein, the annotated NMR data includes: annotated one-dimensional hydrogen NMR spectra; and annotated one-dimensional carbon NMR spectra.

In any one or combination of the embodiments disclosed herein, the annotated NMR data includes one-dimensional NMR data plotted in a space defined by one frequency axis.

In any one or combination of the embodiments disclosed herein, the method further includes training the model based on unlabeled data included in an unlabeled dataset, wherein the training includes: processing, by the model: reference molecular information included in the unlabeled data; and solvent information of a reference solvent associated with the reference molecular information; predicting second NMR shifts associated with the reference molecular information based on processing the reference molecular information and the solvent information of the reference solvent; comparing the second NMR shifts to observed NMR shifts associated with the reference molecular information, wherein the observed NMR shifts are included in the unlabeled data; annotating the unlabeled data based on the comparing; and maintaining or updating the model based at least one of the comparing and the annotating.

In any one or combination of the embodiments disclosed herein, the training of the model based on the unlabeled data is based on an iterative unsupervised learning strategy which includes iterating between the predicting the second NMR shifts, the comparing the second NMR shifts to observed NMR shifts, and the maintaining or updating the model, in association with satisfying one or more criteria.

In any one or combination of the embodiments disclosed herein, the unlabeled dataset is different from an annotated dataset associated with pre-training of the model.

In any one or combination of the embodiments disclosed herein, the unlabeled data includes two-dimensional NMR data plotted in a space defined by two frequency axes.

In any one or combination of the embodiments disclosed herein, the method further includes: predicting heteronuclear single quantum coherence cross peaks associated with the molecular structure and the solvent based on processing the molecular graph and the solvent information.

In any one or combination of the embodiments disclosed herein: the molecular graph is processed in-part by a graph neural network module included in the model; and the solvent information is processed in-part by a solvent encoder included in the model.

Embodiments of the present disclosure are directed to a system including: a processor and a memory, wherein the memory includes instructions stored thereon that, when executed by the processor, cause the processor to perform operations including: processing, by a model: a molecular graph associated with a molecular structure; and solvent information of a solvent associated with the molecular structure; and predicting nuclear magnetic resonance (NMR) shifts associated with the molecular structure based on processing the molecular graph and the solvent information.

In any one or combination of the embodiments disclosed herein, the NMR shifts include at least one of: hydrogen NMR shifts associated with the molecular structure; and carbon NMR shifts associated with the molecular structure.

In any one or combination of the embodiments disclosed herein, the instructions, when executed by the processor, further cause the processor to perform operations including: generating a concatenated representation associated with the molecular structure and the solvent, based on processing the molecular graph and the solvent information; and processing, by one or more multilayer perceptron (MLP) components included in the model, the concatenated representation, wherein predicting the NMR shifts is based on processing the concatenated representation.

In any one or combination of the embodiments disclosed herein, the instructions, when executed by the processor, further cause the processor to perform operations including training the model based on one or more of: annotated NMR data included in an annotated dataset; or unlabeled data included in an unlabeled dataset.

In any one or combination of the embodiments disclosed herein: the molecular graph is processed in-part by a graph neural network module included in the model; and the solvent information is processed in-part by a solvent encoder included in the model.

Embodiments of the present disclosure are directed to a computer program product including a computer readable storage medium having program instructions embodied therewith, the program instructions executable by a processor to cause the processor to perform operations including: processing, by a model: a molecular graph associated with a molecular structure; and solvent information of a solvent associated with the molecular structure; and predicting nuclear magnetic resonance (NMR) shifts associated with the molecular structure based on processing the molecular graph and the solvent information.

The preceding general areas of utility are given by way of example only and are not intended to be limiting on the scope of the present disclosure and appended claims. Additional objects and advantages associated with the compositions, methods, and processes of the present invention will be appreciated by one of ordinary skill in the art in light of the instant claims, description, and examples. For example, the various aspects and embodiments of the invention may be utilized in numerous combinations, all of which are expressly contemplated by the present description. These additional advantages objects and embodiments are expressly included within the scope of the present invention. The publications and other materials used herein to illuminate the background of the invention, and in particular cases, to provide additional details respecting the practice, are incorporated by reference.

### BRIEF DESCRIPTION OF THE DRAWINGS

The accompanying drawings, which are incorporated into and form a part of the specification, illustrate several embodiments of the present invention and, together with the description, serve to explain the principles of the invention. The drawings are only for the purpose of illustrating an embodiment of the invention and are not to be construed as limiting the invention. Further objects, features and advantages of the invention will become apparent from the following detailed description taken in conjunction with the accompanying figures showing illustrative embodiments of the invention.

FIGS. 1A through 1C illustrate aspects of a framework supportive of nuclear magnetic resonance prediction in accordance with one or more embodiments of the present disclosure.

FIG. 2 illustrates aspects of message passing and node representation updates in a GNN layer in accordance with one or more embodiments of the present disclosure.

FIG. 3 illustrates an example of peak prediction and assignment provided by a model in accordance with one or more embodiments of the present disclosure.

FIGS. 4A, 4B, 5, 6, and 7A through 7D illustrate examples of model performance in accordance with one or more embodiments of the present disclosure compared to other approaches.

FIG. 8 illustrates an example of a system in accordance with aspects of the present disclosure.

FIG. 9 illustrates an example flowchart of a method in accordance with one or more embodiments of the present disclosure.

FIG. 10A illustrates a chart indicating an example solvent distribution of nine solvent classes used in a training dataset for training a model for solvent-aware 2D NMR prediction, in accordance with one or more embodiments of the present disclosure.

FIG. 10B illustrates a table of solvent effect on proton shift prediction in accordance with one or more embodiments of the present disclosure.

### DETAILED DESCRIPTION

A detailed description of one or more embodiments supported by aspects of the present disclosure are presented herein by way of exemplification and not limitation with reference to the Figures.

Nuclear magnetic resonance (NMR) spectroscopy has emerged as a versatile tool with widespread applications across diverse scientific domains, including chemistry, environmental science, food science, material science, and drug discovery by unraveling molecular dynamics and structures. In some cases, the primary information of an NMR spectrum arises from the chemical shift, which is determined by the local environment of a nucleus and influenced by interactions through chemical bonds and space. The mechanism of an NMR spectrum yields unique “fingerprints” corresponding to diverse functional groups or molecular motifs, which may facilitate the streamlined deduction of atomic connectivity and arrangement.

Some approaches for interpreting NMR spectra may be based on a set of guidelines, often referred to as “rules of thumb”, in which specific chemical shifts are associated with distinctive functional groups. The determination of molecular structures from varying chemical shifts on NMR spectra generally requires the expertise of experienced organic chemists. To facilitate the interpretation of NMR spectra, significant efforts have been directed towards computational simulation of NMR spectra.

For example, some early computational approaches, such as, for example, Hierarchically Ordered Spherical Environment (HOSE) codes, aim to encapsulate atom neighborhoods in concentric spheres, utilizing a nearest-neighbor approach to predict NMR shift values. Another HOSE approach yields mean absolute errors (MAEs) of 3.52 ppm for carbon (C) NMR and 0.29 ppm for hydrogen (H) NMR on the nmrshiftdb2 dataset, which is a NMR database (web database) for organic structures and their nuclear magnetic resonance (NMR) spectra. The nmrshiftdb2 dataset supports spectrum prediction (e.g., C, H, and other nuclei) as well as searching spectra, structures and other properties.

Concurrently, significant efforts have been devoted to the ab initio calculation of NMR properties. In computational chemistry, ab initio methods may be used to calculate molecular properties using quantum mechanics. In some cases, ab initio methods may be based solely on the basic laws of quantum theory (e.g., solving the Schrödinger equation) to predict the behavior of atoms and molecules, rather than using experimental data or empirical approximations. Examples of ab initio methods include Hartree-Fock and density functional theory (DFT). DFT-based methods have been developed for certain small organic molecules, achieving MAEs of 2.9 ppm for C NMR and 0.23 ppm for H NMR. However, the accuracy of such DFT-based methods may rely heavily on the choice of the basis functions, which may often involve meticulous case-by-case manual tuning for each molecule. Further, for example, the time-intensive nature of DFT calculations may limit the applicability of DFT calculations to comprehensive and large datasets.

Recently, the rise of Graph Neural Networks (GNNs) and the successes of GNNs for predicting molecular properties has prompted initiatives to employ GNNs for predicting peaks in NMR spectra. The application of GNNs to molecules is intuitive, as a molecular structure may be naturally represented as a graph, with each atom represented as a node, and the chemical bonds of the atom represented as edges. However, while considerable efforts have been made in developing predictive models for 1D NMR, the prediction of 2D NMR remains underexplored.

Heteronuclear single quantum coherence (HSQC) spectroscopy, a sophisticated 2D NMR technique, may serve as a tool for elucidating atomic connectivity within complex molecules where conventional 1D NMR prove insufficient. By correlating the chemical shifts of hydrogen nuclei with those of heteronuclear nuclei, typically carbon or nitrogen, via scalar coupling interactions, HSQC facilitates the comprehensive mapping of interatomic connections within a molecule. Such mapping may yield insights into chemical bonding, molecular conformation, and intramolecular interactions. A notable approach in this domain utilizes the ML approach to establish correlations between DFT-simulated HSQC spectra and empirical data to identify molecules. However, the accurate prediction of HSQC spectra using ML techniques remains elusive. Some factors which negatively impact achieving accurate prediction may include: the scarcity of annotated datasets for training ML models, difficulties in handling the inherent sparsity of HSQC spectra which complicates feature representation, the computational demands for achieving accurate full spectrum prediction, and the need for an in-depth comprehension of molecular structures.

Example aspects are described herein which highlight differences between 1D and 2D NMR.

While there are abundant annotated 1D spectra for the training of machine learning models, existing approaches are unable to effectively combine the spectra together to generate reliable 2D NMR data, even under the same experimental conditions. This is primarily because the proton chemical shifts of HSQC or HMQC cross peaks represent ¹³C-bound protons, whereas the main signals in the H represent ¹²C-bound protons, causing the chemical shift values to differ to a varying extent. Typically, in some cases, proper calibration may be required to ensure precise alignment between 1D and HSQC data before interpreting HSQC spectra. Observing discrepancies between 1D and 2D NMR predictions, a recent study introduced a method to integrate proton and carbon 1D spectra into HSQC spectra, achieving mean absolute errors (MAEs) of 0.157 ppm for ¹H and 2.643 ppm for ¹³C. This study underscores the inherent difficulties in accurately predicting 2D NMR chemical shifts, as even though the selected 1D NMR models and methods achieve low error individually, such success cannot be transferred to HSQC cross-peak prediction.

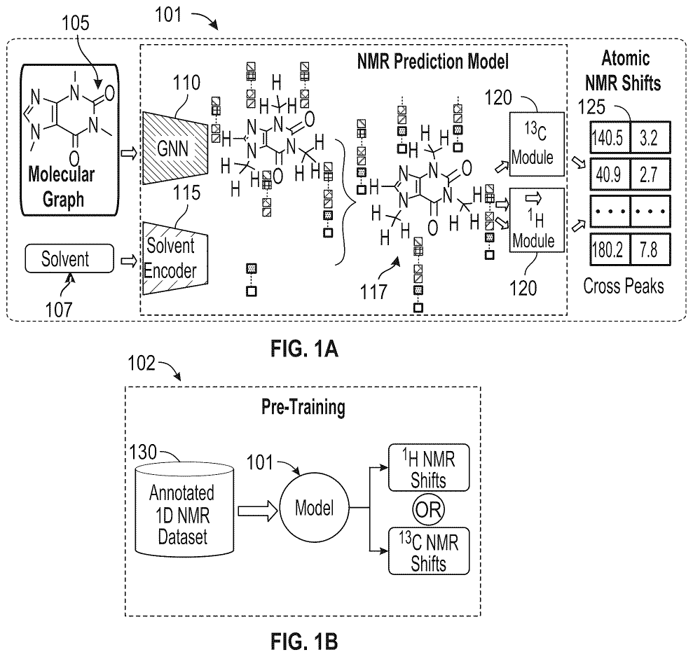

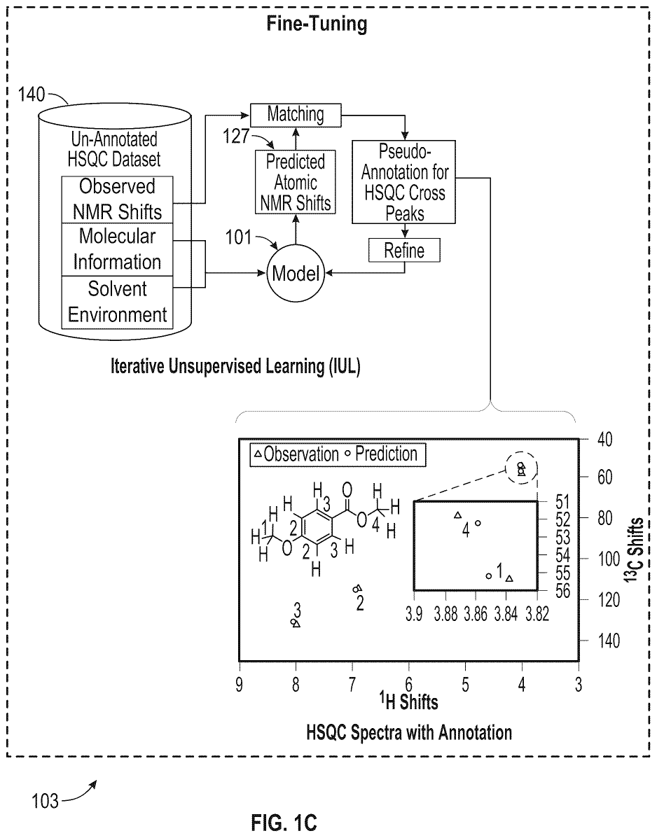

In light of the aforementioned challenges and opportunities in interpreting HSQC spectra, a framework and techniques are provided herein which support a prediction model 101 (also referred to herein as a solvent-aware machine learning model or a machine learning model) designed to predict HSQC cross peaks based on molecular structures, with the capability of peak assignment for experimental spectra, example aspects of which are later described with reference to FIGS. 1A through 1C.

Alongside a GNN module 110 (also referred to herein as a GNN component) capturing structural nuances, the model 101 incorporates a solvent encoder 115 (also referred to herein as a solvent encoder component) configured to effectively account for the impact of solvent environments (e.g., solvents 107) on chemical shifts. Accordingly, for example, the model 101 is capable of delivering accurate cross peak prediction and peak assignment of HSQC spectra.

To tackle the lack of annotated HSQC data, embodiments of the present disclosure support a two-step training process of training the model 101.

At 102, the training process includes pre-training the model 101 on a labeled 1D NMR dataset 130 (also referred to herein as an annotated 1D NMR dataset) via multi-task pre-training (MTT). The pre-training enables the model 101 to learn a wide range of C—H interactions. In some aspects, the model 101 is implemented as a unified model capable of predicting both C shifts and H shifts, in contrast to as two separate models for respectively determining C shifts and H shifts.

At 103, the training process includes implementing an iterative unsupervised learning (IUL) strategy that uses an unlabeled HSQC dataset 140. Using the unlabeled HSQC dataset 140, the training process at 103 includes refining the ability of the model 101 to accurately discern and label HSQC cross peaks. As will be described herein, the model 101 demonstrates superior accuracy across all molecular weight categories compared to other tools (e.g., ChemDraw, Mestrenova).

In some cases, effective and accurate 2D NMR prediction may remain a challenge due to the lack of an annotated 2D NMR training dataset. However, in accordance with one or more embodiments of the present disclosure, to address this gap, the systems and techniques described herein support an iterative unsupervised learning (IUL) approach (at 103) which trains the prediction model 101 for predicting atomic 2D NMR cross peaks and annotating peaks in experimental 2D NMR spectra.

For example, initially at 102, the training process may include implementing a multi-task pre-training (MTT) phase of training the model 101 using a set of annotated 1D H and C NMR spectra. In an example, the set of annotated 1D H NMR spectra and annotated 1D C NMR spectra may be stored in and accessed from the labeled 1D NMR dataset 130.

At 103, the training process may include iteratively improving the model 101 through a fine-tuning process with IUL. The IUL process may include alternating between using the model 101 to annotate the unlabeled HSQC dataset 140 (unlabeled 2D NMR data), thereby generating new annotations, and refining the model 101 using the newly generated annotations.

Example test results achieved using the techniques described herein are provided. The model 101 was trained on 19,000 Heteronuclear Single Quantum Coherence (HSQC) spectra, tested on 500 HSQC spectra with expert annotations, and further compared against two other methods (ChemDraw and Mestrenova) on another expert-annotated HSQC dataset. For HSQC cross peak prediction, the model 101 provided in accordance with one or more embodiments of the present disclosure achieved mean absolute error (MAE) of 2.035 ppm and 0.163 ppm for C shifts and H shifts on the test dataset, respectively, and outperforms the other methods. In accordance with one or more embodiments of the present disclosure, the model 101 demonstrates a capability for accurately predicting chemical shifts, and further, an effectiveness in determining peak assignments for experimental HSQC spectra.

With continued reference to FIGS. 1A through 1C, example aspects are described with respect to the model 101, pretraining (at 102) of the model 101, and fine-tuning (at 103) of the model 101.

The model 101 is configured to derive an atomic representation of a molecular structure (molecular graph 105) by the GNN module 110. The solvent encoder 115 is configured to encode solvent information associated with a solvent 107 into a latent representation.

The model 101 is configured to concatenate (e.g., link) the representation of each atom with the solvent representation, generating a concatenated representation 117. The model 101 is configured to feed the concatenated representation 117 to a MLP component 120 (multilayer perceptron (MLP) component). The model 101 may predict shift data 125 by processing the concatenated representation 117. The shift data 125 may include atomic NMR shifts (e.g., H NMR shifts and/or C NMR shifts described herein).

The model 101 may generate 2D NMR cross peak predictions, example aspects of which are described herein, by generating C—H pair signal predictions in the molecular graph 105. According to chemical knowledge, each carbon can connect to one, two or three protons, creating at most 2 proton signals in an experiment. Using this insight, for each carbon in the molecular graph 105, the model 101 may predict one or more hydrogen signals. In an example, using the described insight, for each carbon in the molecular graph 105, the model 101 may predict at most 2 hydrogen signals, which may aid in distinguishing local structure.

Model pre-training at 102 may include pre-training the model 101 on the annotated 1D NMR dataset 130 using MTT.

Model fine-tuning at 103 may include refining the model 101 (e.g., following pre-training of the model 101) through the IUL process using the unlabeled HSQC dataset 140. Accordingly, for example, the model 101 is configured to provide a final output which includes both HSQC cross peaks and atom alignment.

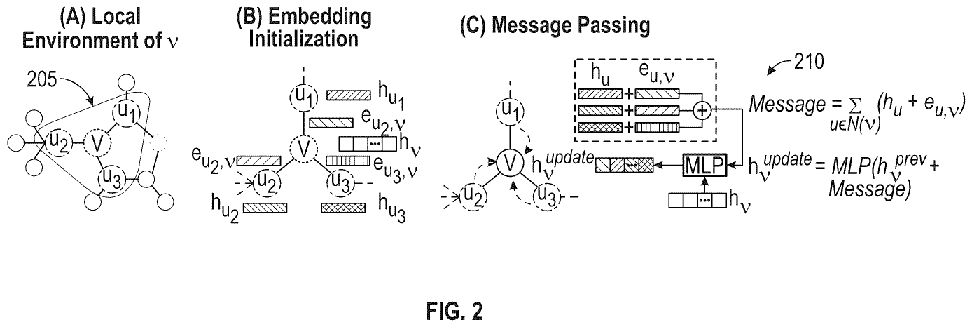

FIG. 2 illustrates aspects of message passing and node representation updates in a GNN layer in accordance with one or more embodiments of the present disclosure. The GNN layer may be implemented at GNN module 110 of FIG. 1A.

As illustrated at (A), for a given center atom in a molecular graph (e.g., molecular graph 105 of FIG. 1A), the local environment 205 contains the neighborhood atom that is directly bonded to the center atom.

As illustrated at (B), for a center node (v), initial random representations are assigned to the center node (v), its neighboring nodes (u1, u2, u3), and their connecting bonds (e_{u1,v}, e_{u2,}_{^{v}}, eᵤ_{^{3,v}}.).

An example of message passing and node update implemented by the GNN layer is described herein with reference to (C). The GNN layer may aggregate and integrate representations of all neighboring nodes and edges to form a message 210 to the center node (v). The GNN layer may then update the representation of the center node (v) to incorporate the message 210 and information from its previous state.

### Methods

Example aspects of the components of the model 101 and the training strategy are further described in detail with reference to FIGS. 1A through 1C and FIG. 2.

### 2D NMR Prediction Model

As illustrated in FIG. 1, the model 101 contains a GNN module 110 for encoding molecular features and a solvent encoder 115 for embedding solvent information. The GNN module 110 learns atomic embeddings that capture both the local and global chemical environments of each atom, which may be beneficial for (and in some cases, be essential) for understanding the observed NMR chemical shifts. The learnt atom representations are expanded by the solvent embedding implemented by the solvent encoder 115. The model 101 may map the resulting concatenated representation 117 to C and H cross peaks by using the MLP component 120.

### GNN

In accordance with one or more embodiments of the present disclosure, a molecule can be represented by a graph G=(V, E), where V is the node set representing atoms and E is the edge set representing chemical bonds. Three features are provided for each node: atomic type, chirality, and hybridization. Also, two features are considered for each edge: bond type and bond direction. Bond types include: Single, Double, Triple, and Aromatic, each reflecting a distinct configuration of electron sharing between atoms.

Bond direction includes None, EndUpRight, and EndDownRight, primarily representing stereochemistry in double bonds. Each atom's feature vector is embedded into a representation vector by a learnable encoder. Similarly, each edge's feature vector is embedded into a representation vector of the same length by another learnable encoder.

Then, the GNN module (GNN model) may utilize the message passing mechanism described with reference to FIG. 2 to iteratively refine the representation of each node based on, for example, information from neighbors of each node and connected edges. The described mechanism allows the learnt node representation to effectively capture structural context, reflecting the foundational principles of atomic interactions.

Example aspects of the implementation of the message passing mechanism of FIG. 2 are further described herein. Embodiments of the message passing mechanism include iterating for a predefined number of layers L, facilitating the propagation of information throughout the graph. Consequently, each node can gradually accumulate information from a wider neighborhood across successive layers. Accordingly, for example, the model 101 (using GNN module 110) is capable of providing final representations of each node, in which each final representation captures both local and global structural information. In some examples, the model 101 features 5 GNN layers, with an atomic embedding dimension of 512.

### Solvent Encoder

In accordance with one or more embodiments of the present disclosure, the incorporation of the solvent encoder 115 into the model 101 supports accurate capture of the influence of a solvent 107 (or multiple solvents 107) on NMR chemical shifts. For example, in some cases, a solvent may have a profound impact on NMR chemical shifts.

In some examples, embodiments of the present disclosure include 9 principal solvent groups which have been identified based on their prevalence in a reference dataset and domain-specific understandings of distinct impacts of the solvent groups on NMR shifts. The principal solvent groups include trichloromethane, dimethyl sulfoxide, acetone, acids, benzene, methanol, pyridine, water, and an additional category to encompass any unspecified solvents from the reference dataset (termed “unknown”).

In accordance with one or more embodiments of the present disclosure, the solvent encoder 115 transforms each discrete solvent group i into a unique, dense feature vector Sa, where d is the embedding dimension. Embodiments of the present disclosure include optimizing these learnable vectors alongside other model parameters during training, resulting in representations that accurately reflect the impact of each solvent class.

Given the different sensitivities of carbon (C) and hydrogen (H) nuclei to solvent environments, embodiments of the present disclosure include choosing a different embedding dimension d to tailor the solvent effect modeling for each nuclei type. For example, a larger embedding dimension d allows the embedding to more effectively capturing the nuanced influence of solvents on NMR shifts. In an example implementation, the solvent embedding dimension d implemented by the solvent encoder 115 is 32, but is not limited thereto.

### Atomic NMR Shift Prediction

As described herein, embodiments of the present disclosure includes concatenating the embedding of each atom h^{(L)} and the solvent embedding S^{d} for each solvent class i to produce a holistic representation of the atom within the context of the molecular structure of the atom and the given solvent. The techniques described herein include subsequently processing the combined representation by MLP component 120 (a MLP network) to predict shift data 125 associated with the atom (NMR shifts for the atom), in accordance with Equation (1):

$${y}_{v} = MLP ( {h}_{v}^{( L )} ⊕ {S}_{i}^{d} ) \qquad ( 1 )$$

where yv is the predicted chemical shift of atom v, h^{(L)} is the atom level embedding produced by GNN, Sd is the solvent embedding, and ⊕ is the concatenation operation. By integrating solvent embedding and atomic embedding, the model 101 effectively combines intrinsic molecular properties and solvent effects, enhancing the ability of the model 101 to predict atomic NMR shifts accurately.

In accordance with one or more embodiments of the present disclosure, two separate MLP components 120 (not illustrated) are used for predicting C and H shift in the cross peak predictions, respectively. Each C atom can bond up to 4 H atoms. When bonded to one, three, or four H atoms, a C atom typically shows only one cross peak in an experimental spectrum. However, when a C atom is connected to two H atoms, up to two cross peaks may be observed, depending on the chiral center. Consequently, a C atom can exhibit at most two C and H cross peaks.

In light of this observation, the model 101 may include a MLP component 120 dedicated to predicting the C shifts and another MLP component 120 for predicting the corresponding H shifts. For cross peak predictions, the C shifts are predicted using the embeddings of C atoms. The corresponding H shift predictions for each C atom incorporate aggregates of embeddings from all bonded H atoms, resulting in two predictions that are typically very similar when one cross peak is theoretically possible.

Aspects of using multiple MLP components 120 described herein enhances the accuracy of the model 101 in predicting H shifts by leveraging the C atom-centered aggregation of the H atom context. By integrating the contextual dynamics around each C atom, the model 101 provides a more detailed and accurate mapping of hydrogen environments, crucial for pinpointing precise cross peaks in complex HSQC spectra. In an example implementation, the model 101 includes 2 MLP layers (e.g., a MLP component 120 dedicated to predicting the C shifts, MLP component 120 for predicting the corresponding H shifts), where the hidden dimension is 128 for the first MLP layer and 64 for the second MLP layer. That is, for example, the model 101 may use a MLP component 120 of dimension [128, 64] for H prediction and use another MLP component 120 of dimension [128, 64] for predicting C shifts. Embodiments of the framework described herein support flexible changing of such dimensions as applicable in association with providing prediction described herein.

### Training Strategy

In some cases, cross peaks may notably be sparse in an HSQC spectrum, where typical resolutions for C and H shifts may be 0.1 and 0.01 ppm, respectively. A typical HSQC spectrum can include 20,000 readings, covering C shifts from 0 to 200 ppm and H shifts from 0 to 10 ppm. However, almost all of these readings are zeros, with only a small fraction representing the potential cross peaks of C—H bonds, crucial for molecular structure analysis. Moreover, the scarcity of annotated HSQC data, particularly the labor-intensive annotations that link cross peaks to C—H bonds, may inhibit effective model training due to lack of data.

In addressing the lack of annotated HSQC data, the techniques described herein include (at 102) deploying MTT to pre-train the model 101 using an extensive annotated 1D NMR dataset 130. The pre-training at 102 acclimates the model 101 with a broad range of molecular structures and their chemical shifts, and enables the model 101 to capture the intricate interplay between molecular structures and their NMR characteristics.

At 103, the techniques described herein include utilizing the IUL strategy to refine the model 101 further on the HSQC dataset 140. The fine-tuning at 103 may include iterative cycles of prediction, annotation, and retraining, progressively enhancing the ability of the model 101 to understand the complex relationships and patterns within the HSQC spectra, and thus the model 101 may have improved predictive accuracy and provide precise cross peak alignments compared to other approaches. For example, other models are unable to provide annotation as described herein.

As described herein with reference to FIGS. 1A through 1C, by combining MTT and IUL, the model 101 is pre-trained and fine-tuned to have annotation capabilities for 2D data (i.e., annotation capabilities are extended from 1D to 2D data). The techniques described herein enhance the predictive power and utility of the model 101 as a robust tool for NMR spectra analysis.

### Pre-Training on 1D NMR Data

In the pre-training phase at 102, embodiments of the present disclosure include utilizing approximately 24,000 annotated 1D NMR data points. Among these, around 22,000 samples exclusively feature C shifts, approximately 400 samples solely exhibit H shifts, while roughly 1,600 samples contain both H and C shifts. To train the model 101 effectively for predicting both H and C shifts, the pre-training phase adapts the MTT approach, which enables simultaneous training on multiple related tasks.

For cases in which the input data contains C shifts, the model 101 predicts only carbon shifts and assesses the errors between the predicted and actual values. Conversely, for cases in which the data sample contains H shifts, the hydrogen shift prediction module is activated, and the model 101 predicts only hydrogen shifts and assesses the errors between the predicted and actual values. In both scenarios, embodiments of the present disclosure include updating the embeddings of C and H atoms in the GNN module 110 simultaneously, benefiting from the message passing mechanism.

Therefore, the learnt representations may contain an understanding of C—H relationships (e.g., an indirect understanding of C—H relationships) and support the interpretation of HSQC data as described herein. However, in some examples, the relative scarcity of H shift data, due to the difficulties in accurately obtaining, extracting, and aggregating peaks H from experimental data, may complicate the training process as focusing extensively on one type of shift (e.g., C shifts) could compromise the ability of the model 101 to accurately predict the other (e.g., H shifts).

To address the problem of a lack of available shift data in the MTT training, embodiments of the present disclosure include performing over-sampling on a subset of data that contain both H and C shifts, and those containing only H shifts. Consequently, the learned representations develop a fundamental understanding of C—H relationships, thus supporting effective interpretation of HSQC data. The integration of learned atomic relationships streamlines the transition to HSQC cross peak predictions, thereby enhancing the accuracy and efficiency of the model 101 in analyzing HSQC spectra.

### Unsupervised Fine-Tuning on HSQC Data

The model 101 pre-trained on the 1D NMR dataset 130 is further trained at 103 to provide an improved ability to predict HSQC cross-peaks from molecular structures.

First, the fine-tuning at 103 includes training the model 101 to account for substantial differences between the nature of 1D NMR data and HSQC data. That is, for example, the chemical shifts observed in HSQC cross peaks often do not correlate directly with their counterparts in 1D NMR spectra, particularly regarding H shifts. For example, in the 1D NMR data, the chemical shifts of non-singlet peaks are averaged as ground truth, lacking the precision involved with HSQC spectroscopy. Also, the variations in relaxation properties, coupling effects, and fluctuations in the local magnetic field can all contribute to the distinctions between 1D NMR and HSQC peaks. Moreover, experimental conditions and pulse sequences used in HSQC experiments can introduce slight deviations in chemical shift values compared to those observed in the H spectrum.

Second, the fine-tuning at 103 includes training the model 101 to account for substantial differences between molecule distributions in 1D NMR data (included in 1D NMR dataset 130) and HSQC data (included in unlabeled HSQC dataset 140). In an example implementation, the HSQC dataset 140 includes 76.34% small molecules and 90.33% non-saccharides, whereas the 1D NMR dataset 130 includes 98.80% small molecules and 99.95% non-saccharides. The fine-tuning at 103 refines model 101 (which is pre-trained at 102 for NMR prediction) on the HSQC dataset 140.

Third, in some embodiments, solvent environment information is not included in the 1D NMR dataset 130, but since the solvent environment information is available in the unlabeled HSQC dataset 140, such impact may be incorporated in the techniques described herein. The shift of H is much more sensitive in a change of solvent (e.g., the shift of H may have a relatively high sensitivity to changes in solvent), and the model 101 is configured to address such sensitivity.

In some aspects, the HSQC dataset 140 is not annotated. Accordingly, for example, the fine-tuning at 103 implements an IUL training strategy which iterates between (a) aligning cross peak prediction from the model 101 with the experiment observations to annotate the HSQC data and (b) using the newly acquired annotations to fine-tune the NMR prediction model, until convergence.

### Pseudo-Annotation of HSQC, as Provided by the Model 101

At the end of each round in the IUL process implemented at 103, the techniques described herein include aligning signals predicted by the model 101 with the experimental observations to create pseudo-labels. For example, the techniques described herein may include aligning or matching atomic NMR shifts 127 predicted by the model 101 with observed NMR shifts obtained from the unlabeled HSQC dataset 140.

In an example where the number of C—H bonds in a molecular graph 105 matches the observed HSQC cross peaks, the aligning may include using the Hungarian optimization algorithm. The Hungarian optimization technique solves assignment problems by minimizing the cost of matching a set of predictions to a set of observations. In the context of NMR analysis described herein, the “cost” is defined as the discrepancy between the predicted chemical shifts and the actual shifts observed experimentally. By systematically reducing these differences, the Hungarian algorithm achieves an optimal one-to-one correspondence between predicted shift pairs and experimental signals, even in complex scenarios with potential signal overlap.

In an example in which the number of C—H bonds within a molecule (molecular graph 105) exceeds the number of signals recorded, peak alignment may be more difficult. This mismatch in numbers may arise from several factors: firstly, rotational equivalence can reduce the number of signals, with a single peak representing all three C—H bonds for methyl groups; secondly, symmetrical molecular structures can result in a single detectable signal for multiple symmetric C—H bonds, as seen in benzene molecule where only one peak represents all six C—H bonds; lastly, in highly complex molecules, overlapping signals obscure some peaks, reducing the detectability of individual C—H bonds from experiments.

To overcome the described difficulties associated with peak alignment and mismatches, the fine-tuning at 103 may include utilizing the graduated assignment algorithm (e.g., to iteratively refine the predictive capabilities of the model 101), which facilitates matching between graphs of different node counts, making the graduated assignment algorithm particularly suitable for this scenario.

In the graduated assignment algorithm, the C—H shifts (C^{l},H^{l})ᵢ₌₀^{N} predicted by the model 101 and the observed C—H signals (C^{j},H^{j})ⱼ₌₀^{M} for each molecule are conceptualized as points on a 2D plane, where N and M are numbers of predicted and observed C—H shifts respectively. The techniques described herein include treating the points as vertices in two fully connected graphs, with G₁ for predicted shifts and G₂ for observed signals. The similarity between nodes is defined as the inverse of differences between predicted chemical shifts (node in G₁) and observed chemical shifts (node in G₂). Specifically, for each predicted shift, the techniques described herein include computing the difference between the predicted shift and a corresponding observed shift, where a smaller difference indicates a higher similarity.

To derive an assignment matrix A where each element Aᵤᵥ∈{0, 1} indicates whether node u in G₁ matches with node v in G₂, the graduated assignment algorithm first finds the soft matching matrix that relaxes the binary constraint Aᵤᵥ∈{0, 1} to a continuous range [0, 1], and then converts the soft matching matrix into hard assignment in a greedy way, enabling one-to-many matching.

Example results and implementation details associated with pre-training and fine tuning of the model 101 in accordance with one or more embodiments of the present disclosure are now further described herein.

### Manual Annotation

In contrast to pseudo annotation described herein, a manual annotation process may be deployed on a small set of test data to evaluate model performance. In some cases, a manual annotation process may involve three experienced experts with extensive knowledge in organic chemistry. For each molecule, two experts may independently link the observed cross-peaks from experiments to C—H bonds. If the two experts agree, the annotation is finalized. In cases of disagreement, the third expert may review and validate the annotations. In manual annotation, samples with poor quality, such as those with insufficient experimental resolution, are excluded from the test dataset for model evaluation.

### Data

In an example implementation, the MTT process at 102 may use, as the pre-training dataset, a 1D NMR dataset 130 from NMRShiftDB2, which contains ˜24,000 annotated NMR spectra collected from 22,663 distinct molecules. In an example implementation, the datasets used in the IUL process at 103 include a training dataset containing ˜19,000 experimental HSQC spectra and a validation dataset containing ˜5,000 HSQC spectra, collected from HMDB and CH-NMR-NP. To quantitatively evaluate the model 101, the techniques described herein include building a test dataset by randomly selecting 500 spectra and manually annotating them to establish the ground truth. These 500 spectra were randomly divided into 5 subsets of 100 spectra each, which supports conducting the assessment 5 times and evaluating the variation in performance. Example results comparing aspects of the model 101 in accordance with one or more embodiments of the present disclosure to other prediction approaches in chemistry are further described in the attached appendix.

### Performance on HSQC Cross Peak Prediction and Assignment

Table 1 summarizes the performance of the model 101 on the tasks of HSQC cross peak prediction and peak assignment using the 1st test dataset, in accordance with one or more embodiments of the present disclosure. Specifically, the model 101 achieves an MAE of 2.05 ppm for C shifts and 0.165 ppm for H shifts. The robustness of the approach described herein results in exceptionally high accuracy for peak assignments at both the molecular and peak levels, achieving 95.21% and 81.56% respectively. The model 101 is said to be correct on a molecule if all cross peaks of the molecule are correctly assigned.

Regarding performance on HSQC cross peak prediction and peak assignment for the 1st test set, table 1a reports the Mean Absolute Error (MAE), and table 1b reports the accuracy of peak assignments (the manual annotations) produced by the approaches described herein. The numbers in the parentheses are the standard deviations.

TABLE 1(a)
Model Performance
MAE (ppm)

| ¹³C Shift | ¹H Shift |
|---|---|
| 2.05 | 0.165 |

TABLE 1(b)
Peak Assignment Accuracy

| | Peak Accuracy (%) |
|---|---|
| Fully-Correct | for Partial-Correct |
| Molecule (%) | Molecule |
| 95.21% | 81.56% |

The peak level accuracy is calculated as the ratio of correctly assigned cross peaks to the total number of cross peaks. In terms of annotation accuracy, for a test case of the model 101, the model 101 accurately annotated all peaks in 456 out of 479 molecules (95.21%). For those 23 molecules for which algorithmic annotations provided by the model 101 do not fully agree with the experts, 81.56% of the peak annotations still align.

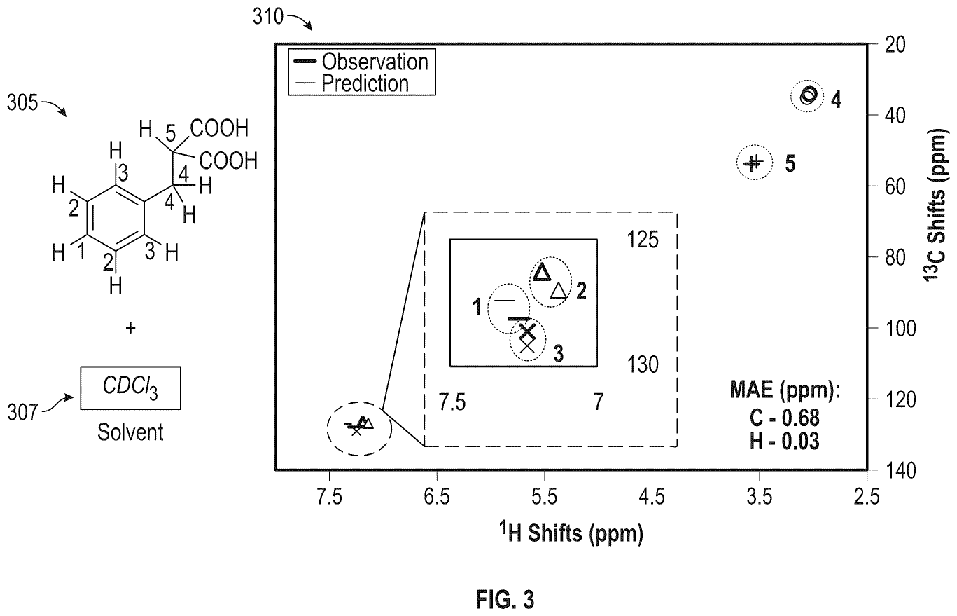

FIG. 3 illustrates an example of peak prediction and assignment which may be provided by the model 101, using inputs of a molecule 305 and solvent 307 in accordance with one or more embodiments of the present disclosure.

FIG. 3 illustrates an example of using the model 101 to accurately predict cross peaks and align them with experimental signals. Referring to the molecule 305 shown at the top-left, each C—H bond is labeled with a numerical identifier. Notably, the symmetric pairs of bonds (labeled as “2”, “3”, and “4”) are each expected to generate a single HSQC cross peak due to their structural equivalence. The predicted HSQC cross peaks (in orange) as provided by the model 101 and the alignments to the experimental observations (in blue) are plotted at 310. The alignments are indicated by the dashed circles.

Comparison with Other Tools

In organic chemistry, simulating HSQC spectra may be crucial for analyzing experimental HSQC spectra, as such simulation assists researchers in assigning the observed cross peaks to the C—H bonds in target molecules. In comparing the model 101 to other software solutions (e.g., ChemDraw and Mestrenova) on the second test dataset (e.g., unlabeled HSQC dataset 140), the results (see Table 2) clearly demonstrate the superiority of the model 101.

Table 2 provides a performance comparison between the model 101 proposed in accordance with one or more embodiments of the present disclosure (indicated at ML Model MAE in Table 2) and established traditional tools on the second test dataset. The model 101 performs better across all molecular weight categories.

TABLE 2

| Molecular | ML Model MAE | | ChemDraw MAE | | Mestrenova MAE | |
|---|---|---|---|---|---|---|
| Weight | ¹³C | ¹H | ¹³C | ¹H | ¹³C | ¹H |
| 0-499 | 1.489 | 0.156 | 1.624 | 0.253 | 1.485 | 0.197 |
| | (0.365) | (0.014) | (0.588) | (0.033) | (0.548) | (0.027) |
| 500-999 | 1.818 | 0.085 | 3.105 | 0.237 | 2.336 | 0.150 |
| | (0.993) | (0.045) | (1.277) | (0.080) | (1.033) | (0.072) |
| 1000+ | 3.780 | 0.287 | 8.859 | 0.640 | 7.714 | 0.602 |
| | (2.239) | (0.108) | (3.452) | (0.297) | (6.870) | (0.421) |
| Overall | 2.424 | 0.176 | 4.319 | 0.377 | 3.863 | 0.307 |
| | (0.525) | (0.037) | (1.213) | (0.080) | (1.959) | (0.146) |

### Effects of Pre-Training and Fine-Tuning

Table 3 compares the ability of the model 101 to predict HSQC cross peaks at different training stages, highlighting the contributions of the training strategy described herein.

After pre-training via MTT on the 1D NMR dataset 130, the model 101 achieves a validation performance with mean absolute errors (MAEs) of 0.210 ppm for H NMR prediction and 2.228 ppm for C NMR prediction. This success can be attributed to MTT, via which the model 101 effectively learns atomic latent features as well as local structural information by simultaneously performing H and C NMR shift predictions. Such effective learning provided by MTT as described herein provides a technological benefit which overcomes the problem with limited annotated HSQC data.

In some examples, following pre-training, the model 101 is able to predict HSQC cross peaks with reasonable MAEs of 1.397 ppm and 2.822 ppm for H and C shifts, respectively. These relatively large MAEs are expected as the data distribution of the unlabeled HSQC dataset 140 (76.34% small molecules and 90.33% non-saccharides) differs significantly from that of the 1D NMR dataset 130 (98.80% small molecules and 99.95% non-saccharides). In addition, the HSQC cross peaks involve interactions beyond simple pairings of 1D C and H shifts, which may involve a deeper understanding of interactions between atoms.

In some aspects, the frequent absence of solvent labels in the 1D NMR dataset 130 may prevents the model 101 from learning solvent effects. Nevertheless, the pre-training via MTT offers a robust foundation for fine-tuning the model 101 using IUL (at 103). Each IUL iteration, may reduce model errors associated with the model 101.

In some aspects, the IUL iterations may provide performance improvements which are relatively higher during the initial IUL iterations and gradually diminish. In an example, by the fifth IUL iteration, the improvement may indicate the convergence of fine-tuning based on the amount of performance improvement compared to the fourth IUL iteration.

In an example, the model 101, following fine-tuning described with reference to 103, may achieve MAEs of 0.165 ppm and 2.05 ppm for H and C shifts, respectively. Throughout the IUL process at 103, the techniques described herein may include training the model 101 to gain a more profound understanding of solvent effects and complex C—H interactions due to intricate molecular structures.

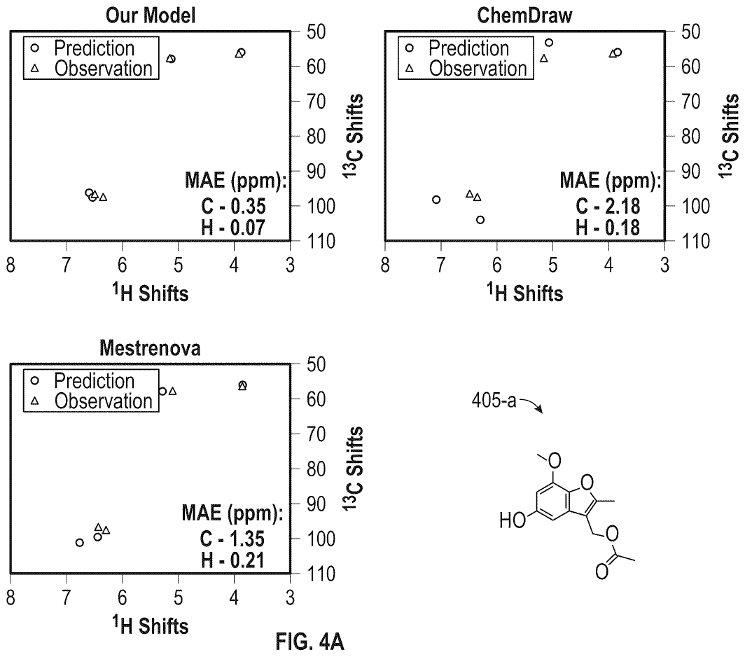

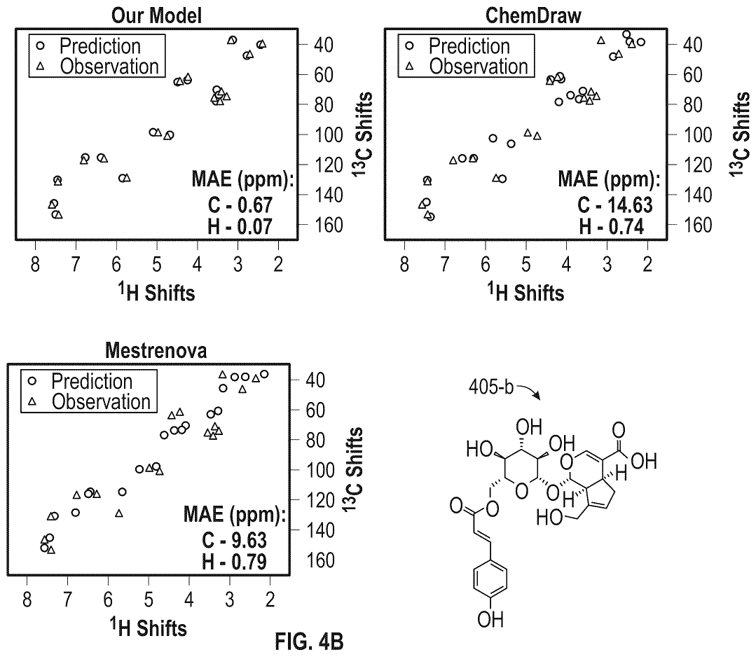

FIGS. 4A, 4B, 5, 6, and 7A through 7D illustrate additional examples of model performance in accordance with one or more embodiments of the present disclosure compared to other approaches.

FIGS. 4A and 4B compare the model 101 provided in accordance with one or more embodiments of the present disclosure to ChemDraw and Mestrenova on two typical examples. A small molecule 405-a with weight of ˜250 Dalton and a larger molecule 405-b with weight of ˜500 Dalton. The observed experimental signals and the predicted signals are colored in blue and orange, respectively. The prediction error (MAE) is shown in the bottom right corner of each plot. The model 101 provides improved performance compared to ChemDraw and Mestrenova, and particularly excels in handling large molecules with complex conformations.

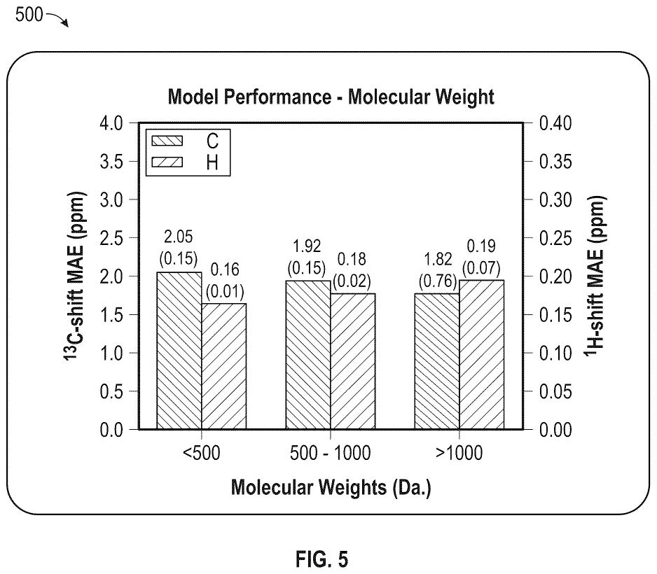

FIG. 5 illustrates a plot 500 comparing performance of the model 101 between small, medium, and large molecules by molecular weight. The model 101 performs equally well between small and medium molecules, with a marginally reduced precision for larger molecules.

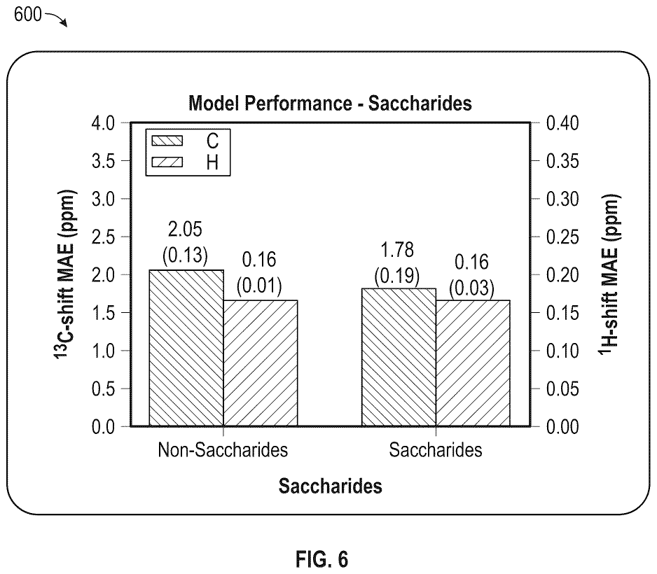

FIG. 6 illustrates a plot 600 comparing performance of the model 101 between saccharides and nonsaccharides. The model 101 performs equally well in both groups.

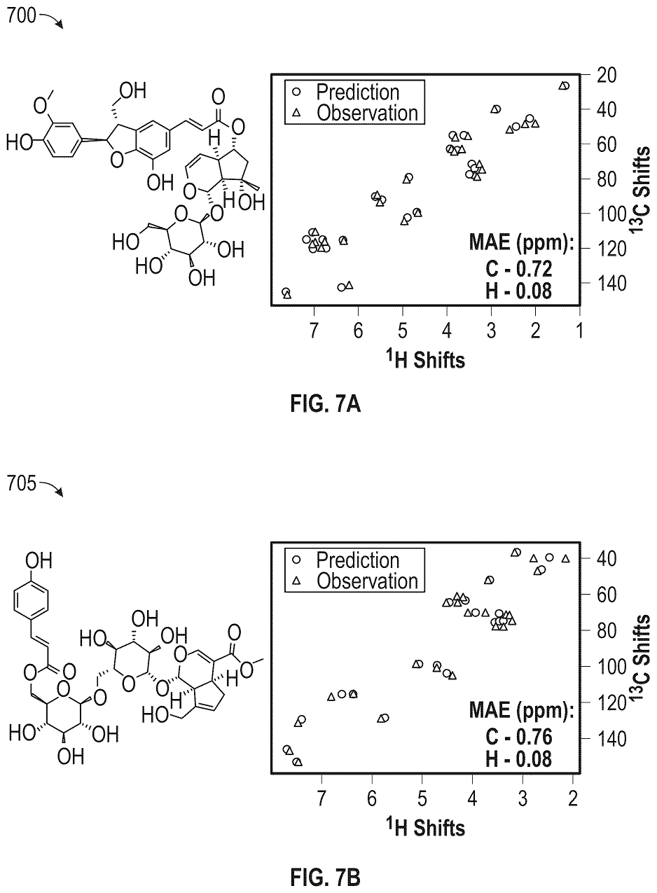

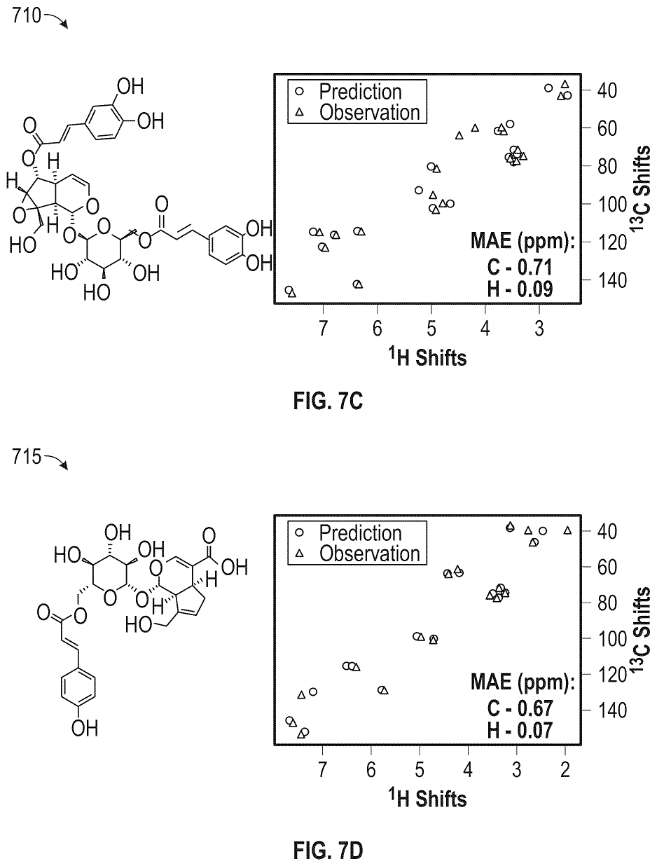

FIGS. 7A through 7D illustrates example plots 700 through 715 of performance of the model 101 on saccharides. The observed experimental signals and the predicted signals are colored in blue and orange, respectively. The prediction error (MAE) is shown in the bottom right corner within each plot.

Additional information associated with FIGS. 4A, 4B, 5, 6, and 7A through 7D is provided in the attached appendix.

As has been described herein, a novel framework is provided for developing machine learning techniques for predicting C—H cross peaks in HSQC spectra. The framework enables tackling of two major challenges in this avenue. The first challenge is the scarcity of annotated HSQC data for training machine learning models. The second challenge is that collecting large volumes of annotated HSQC data is labor intensive and involves highly trained personnel.

In implementing the framework described herein, a model 101 combining a GNN module 110 with a solvent encoder 115 is provided. The GNN module 110 is trained to generate atomic embeddings that encapsulate both the local and global chemical environments of each atom, which supports accurate chemical shift predictions. The atomic embeddings are combined with the solvent embedding produced by the solvent encoder 115, which supports the capability of the model 101 to learn the influence of a solvent on chemical shifts. The combined embeddings are mapped by one or more MLP components 120 to HSQC chemical shifts.

The framework employs a two-stage transductive strategy to train the model 101 while addressing the aforementioned challenges. In the first stage, at 102 of FIG. 1B, a large amount of annotated 1D NMR data is used to pre-train the model 101 via multi-task learning. The pre-training enables the model 101 to adeptly grasp the intricate relationship between atomic interactions and NMR signals, laying a robust foundation for the subsequent stage described with reference to 103 of FIG. 1C.

At 103, the model 101 is refined on a set of unlabeled HSQC spectra via IUL, enhancing the capability of the model 101 in predicting and interpreting HSQC spectra. The model 101, resulting from the pre-training at 102 and IUL implemented at 103, achieves MAEs of 0.165 ppm and 2.05 ppm for H and C shifts respectively, while accurately assigning cross peaks. The model 101 demonstrates a consistent performance across various molecular weight and saccharide categories, significantly outperforming the traditional methods, and shows convincing generalization capabilities to less represented samples from the training dataset.

Further embodiments of the present disclosure may include refining the model 101 by developing 3D-GNN models that are able to consider 3D structural information such as spatial orientation and conformational flexibility. The enhancement supports handling of other 2D NMR spectra, such as correlation spectroscopy and nuclear Overhauser effect spectroscopy, thus broadening the applicability of the model 101 and making further contribution to the field of chemical analysis.

As has been described herein, the techniques described herein differs and provides technical improvements compared to other approaches, in at least three prospectives: 1) usage in elucidating/verifying molecular structures, 2) annotation capability, and 3) working on 2D NMR.

Existing approaches either reconstruct molecular structures from NMR spectra or encode a given NMR spectrum into a representation that is then used to search a pre-established library of molecules. In contrast, the model 101 is capable of directly predicting the cross peaks in the spectrum from the simple molecular line entry system (SMILES) representation, allowing for direct comparison with experimental spectra to verify the correctness of the molecular structure.

Second, besides an accurate prediction of the 2D spectra, the model 101 may provide atom level annotation simultaneously, a functionality absent from other approaches. The atom-level annotations of HSQC spectra provide a powerful means for verifying molecular structures. The techniques described herein support correlating each carbon-hydrogen (C—H) signal in the HSQC spectrum with specific atoms in the molecule, via which researchers can elucidate fine structures of molecules.

Other approaches merely provide models which are trained using simulated 1D NMR data, and the models are not sufficiently tested on experimental data, limiting the creditability of the models. The teachings described herein used experimental 2D NMR data to train and test the model 101, providing better evidence to support the training and performance of the model 101.

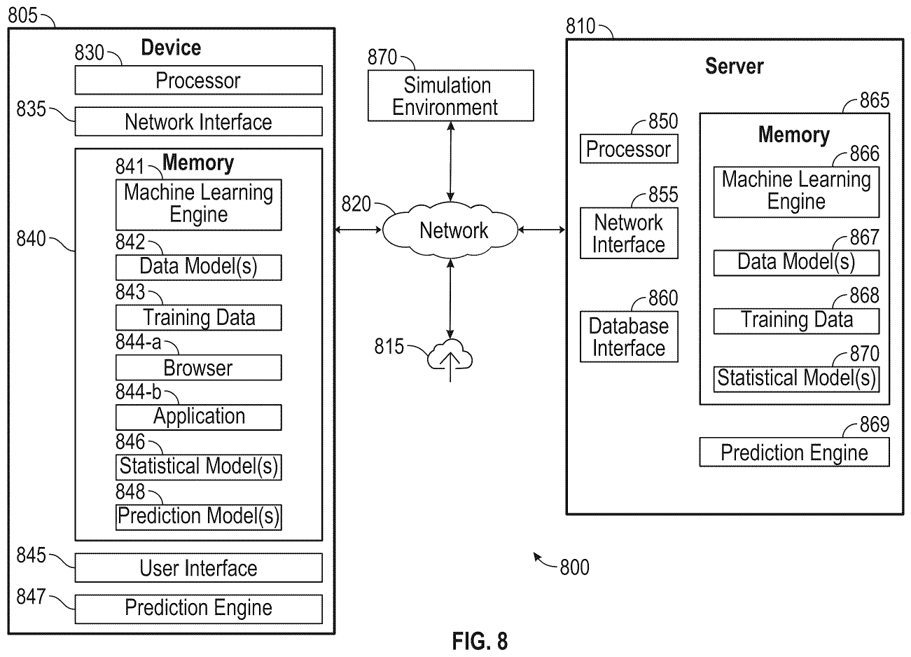

FIG. 8 illustrates an example of a system 800 in accordance with aspects of the present disclosure. The system 800 may include a device 805, a server 810, a database 815, a communication network 820, and a simulation environment 870. The devices 805, the server 810, the database 815, the communications network 820, and the simulation environment 870 may implement aspects of the present disclosure described herein. The device 805 may implement aspects of the techniques and features described herein.

In various aspects, settings of any of the device 805, the server 810, database 815, and the network 820 may be configured and modified by any user and/or administrator of the system 800. Settings may include thresholds or parameters described herein, as well as settings related to how data is managed. Settings may be configured to be personalized for one or more devices 805, users of the devices 805, and/or other groups of entities, and may be referred to herein as profile settings, user settings, or organization settings. In some aspects, rules and settings may be used in addition to, or instead of, parameters or thresholds described herein. In some examples, the rules and/or settings may be personalized by a user and/or administrator for any variable, threshold, user (user profile), device 805, entity (e.g., patient), or groups thereof.

A device 805 may include a processor 830, a network interface 835, a memory 840, and a user interface 845. In some examples, components of the device 805 (e.g., processor 830, network interface 835, memory 840, user interface 845) may communicate over a system bus (e.g., control busses, address busses, data busses) included in the device 805. In some cases, the device 805 may be referred to as a computing resource.

In some cases, the device 805 may transmit or receive packets to one or more other devices (e.g., another device 805, the server 810, the database 815, the simulation environment 870) via the communication network 820, using the network interface 835. The network interface 835 may include, for example, any combination of network interface cards (NICs), network ports, associated drivers, or the like. Communications between components (e.g., processor 830, memory 840) of the device 805 and one or more other devices (e.g., another device 805, the database 815) connected to the communication network 820 may, for example, flow through the network interface 835.

The processor 830 may correspond to one or many computer processing devices. For example, the processor 830 may include a silicon chip, such as a FPGA, an ASIC, any other type of IC chip, a collection of IC chips, or the like. In some aspects, the processors may include a microprocessor, CPU, a GPU, or plurality of microprocessors configured to execute the instructions sets stored in a corresponding memory (e.g., memory 840 of the device 805). For example, upon executing the instruction sets stored in memory 840, the processor 830 may enable or perform one or more functions of the device 805.

The processor 830 may utilize data stored in the memory 840 as a neural network (also referred to herein as a machine learning network). The neural network may include a machine learning architecture. In some aspects, the neural network may be or include an artificial neural network (ANN), a graph neural network (GNN), and the like. In some other aspects, the neural network may be or include any machine learning network such as, for example, a deep learning network, a convolutional neural network, or the like. Some elements stored in memory 840 may be described as or referred to as instructions or instruction sets, and some functions of the device 805 may be implemented using machine learning techniques.

The memory 840 may include one or multiple computer memory devices. The memory 840 may include, for example, Random Access Memory (RAM) devices, Read Only Memory (ROM) devices, flash memory devices, magnetic disk storage media, optical storage media, solid state storage devices, core memory, buffer memory devices, combinations thereof, and the like. The memory 840, in some examples, may correspond to a computer readable storage media. In some aspects, the memory 840 may be internal or external to the device 805.

The memory 840 may be configured to store instruction sets, neural networks, and other data structures (e.g., depicted herein) in addition to temporarily storing data for the processor 830 to execute various types of routines or functions. For example, the memory 840 may be configured to store program instructions (instruction sets) that are executable by the processor 830 and provide functionality of a machine learning engine 841 and a prediction engine 847 described herein. The memory 840 may also be configured to store data or information that is useable or capable of being called by the instructions stored in memory 840. One example of data that may be stored in memory 840 for use by components thereof is a data model(s) 842 (also referred to herein as a neural network model), training data 843 (also referred to herein as a training data and feedback), statistical models 846, and/or other prediction models 848.

The machine learning engine 841 may include a single or multiple engines. The device 805 (e.g., the machine learning engine 841) may utilize one or more data models 842 for recognizing and processing information obtained from other devices 805, the server 810, the database 815, and the simulation environment 870. In some aspects, the device 805 (e.g., the machine learning engine 841) may update one or more data models 842 based on learned information included in the training data 843. In some aspects, the machine learning engine 841 and the data models 842 may support forward learning based on the training data 843. The machine learning engine 841 and data models 842 may support reinforcement learning (e.g., deep reinforcement learning) and imitation learning described herein. The machine learning engine 841 may have access to and use one or more data models 842. For example, the data model(s) 842 may be built and updated by the machine learning engine 841 based on the training data 843. The data model(s) 842 may be provided in any number of formats or forms. Non limiting examples of the data model(s) 842 include Decision Trees, Support Vector Machines (SVMs), Nearest Neighbor, and/or Bayesian classifiers.

The engines described herein (e.g., machine learning engine 841, prediction engine 847) may create, select, and execute processing decisions as described herein. Processing decisions may be handled automatically by the engines (e.g., machine learning engine 841, prediction engine 847), with or without human input.

The engines (e.g., machine learning engine 841, prediction engine 847) may store, in the memory 840 (e.g., in a database included in the memory 840), historical information. Data within the database of the memory 840 may be updated, revised, edited, or deleted by the engines described herein. In some aspects, the engines described herein may support continuous, periodic, and/or batch fetching of data and data aggregation.

The device 805 may render a presentation (e.g., visually, audibly, using haptic feedback, etc.) of an application 844 (e.g., a browser application 844-a, an application 844-b). The application 844-b may be an application associated with executing, controlling, and/or monitoring the simulation environment 870 described herein. For example, the application 844-b may enable control of the device 805 or the simulation environment 870.

In an example, the device 805 may render the presentation via the user interface 845. The user interface 845 may include, for example, a display (e.g., a touchscreen display), an audio output device (e.g., a speaker, a headphone connector), or any combination thereof. In some aspects, the applications 844 may be stored on the memory 840. In some cases, the applications 844 may include cloud based applications or server based applications (e.g., supported and/or hosted by the database 815 or the server 810). Settings of the user interface 845 may be partially or entirely customizable and may be managed by one or more users, by automatic processing, and/or by artificial intelligence.

In an example, any of the applications 844 (e.g., browser application 844-a, application 844-b) may be configured to receive data in an electronic format and present content of data via the user interface 845. For example, the applications 844 may receive data from another device 805, the server 810, or the simulation environment 870 via the communications network 820, and the device 805 may display the content via the user interface 845.

The database 815 may include a relational database, a centralized database, a distributed database, an operational database, a hierarchical database, a network database, an object oriented database, a graph database, a NoSQL (non-relational) database, etc. In some aspects, the database 815 may store and provide access to, for example, any of the stored data described herein.

The server 810 may include a processor 850, a network interface 855, database interface instructions 860, and a memory 865. In some examples, components of the server 810 (e.g., processor 850, network interface 855, database interface instructions 860, memory 865) may communicate over a system bus (e.g., control busses, address busses, data busses) included in the server 810. The processor 850, network interface 855, and memory 865 of the server 810 may include examples of aspects of the processor 830, network interface 835, and memory 840 of the device 805 described herein.

For example, the processor 850 may be configured to execute instruction sets stored in memory 865, upon which the processor 850 may enable or perform one or more functions of the server 810. In some aspects, the processor 850 may utilize data stored in the memory 865 as a neural network. In some examples, the server 810 may transmit or receive packets to one or more other devices (e.g., a device 805, the database 815, another server 810) via the communication network 820, using the network interface 855. Communications between components (e.g., processor 850, memory 865) of the server 810 and one or more other devices (e.g., a device 805, the database 815, the simulation environment 870, etc.) connected to the communication network 820 may, for example, flow through the network interface 855.

In some examples, the database interface instructions 860 (also referred to herein as database interface 860), when executed by the processor 850, may enable the server 810 to send data to and receive data from the database 815. For example, the database interface instructions 860, when executed by the processor 850, may enable the server 810 to generate database queries, provide one or more interfaces for system administrators to define database queries, transmit database queries to one or more databases (e.g., database 815), receive responses to database queries, access data associated with the database queries, and format responses received from the databases for processing by other components of the server 810.

The memory 865 may be configured to store instruction sets, neural networks, and other data structures (e.g., depicted herein) in addition to temporarily storing data for the processor 850 to execute various types of routines or functions. For example, the memory 865 may be configured to store program instructions (instruction sets) that are executable by the processor 850 and provide functionality of the machine learning engine 866 and a prediction engine 869 described herein. One example of data that may be stored in memory 865 for use by components thereof is a data model(s) 867 (also referred to herein as a neural network model) and/or training data 868. The data model(s) 867 and the training data 868 may include examples of aspects of the data model(s) 842 and the training data 843 described with reference to the device 805. For example, the server 810 (e.g., the machine learning engine 866) may utilize one or more data models 863 for recognizing and processing information obtained from devices 805, another server 810, the database 815, or the simulation environment 870. In some aspects, the server 810 (e.g., the machine learning engine 866) may update one or more data models 863 based on learned information included in the training data 868.

In some aspects, components of the machine learning engine 866 may be provided in a separate machine learning engine in communication with the server 810.

The prediction engine 847 and prediction engine 869 may support the prediction techniques described herein. Example aspects of the techniques performable by the prediction engine 847 and the prediction engine 869 are described herein with reference to the following figures. The prediction engine 847 and the prediction engine 869 may be implemented using one or more models (e.g., model(s) 842, statistical models 846, prediction models 848, and the like) described herein.

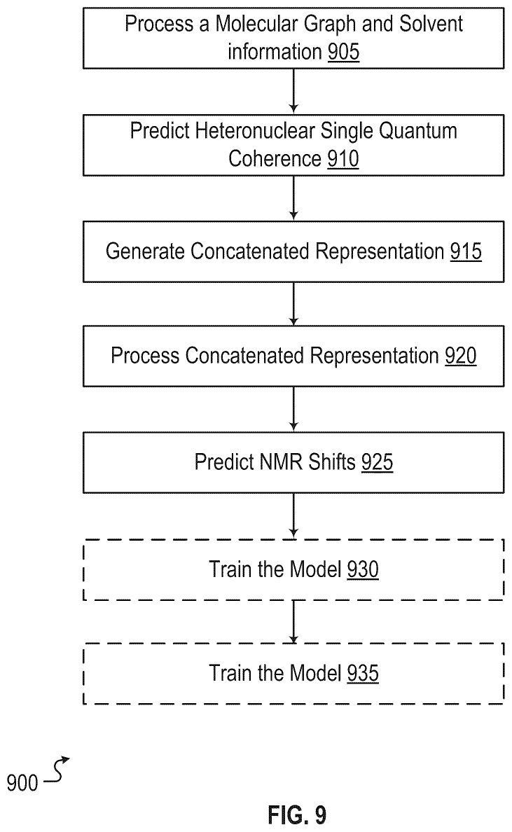

FIG. 9 illustrates an example flowchart of a method 900 in accordance with one or more embodiments of the present disclosure. The method 900 may be implemented by the example aspects of a system 800 or device (e.g., device 805, processor 830, server 810, processor 850) as described herein.

At block 905, the method 900 includes processing, by a model: a molecular graph associated with a molecular structure; and solvent information of a solvent associated with the molecular structure.

In some aspects, the method 900 includes processing the molecular graph in-part by a graph neural network module included in the model; and the method 900 includes processing the solvent information in-part by a solvent encoder included in the model.

At block 910, the method 900 includes predicting heteronuclear single quantum coherence cross peaks associated with the molecular structure and the solvent based on processing the molecular graph and the solvent information.

At block 915, the method 900 includes generating a concatenated representation associated with the molecular structure and the solvent, based on processing the molecular graph and the solvent information.

At block 920, the method 900 includes processing, by one or more multilayer perceptron (MLP) components included in the model, the concatenated representation.

In some aspects, the one or more MLP components may include: a first MLP component configured to predict carbon NMR shifts associated with the molecular structure; and a second MLP component configured to predict hydrogen NMR shifts associated with the molecular structure.

At block 925, the method 900 includes predicting nuclear magnetic resonance (NMR) shifts associated with the molecular structure based on processing the molecular graph and the solvent information.

In some aspects, predicting the NMR shifts is based on processing the concatenated representation.

In some aspects, the NMR shifts may include at least one of: hydrogen NMR shifts associated with the molecular structure; and carbon NMR shifts associated with the molecular structure.

The method 900 may include training the model.

For example, at block 930, the method 900 may include training the model based on annotated NMR data included in an annotated dataset.

In some aspects, training the model at block 930 may include multi-task pre-training of the model based on the annotated NMR data.

In some aspects, the annotated NMR data may include: annotated one-dimensional hydrogen NMR spectra; and annotated one-dimensional carbon NMR spectra.

In some aspects, the annotated NMR data may include one-dimensional NMR data plotted in a space defined by one frequency axis.

In another example, at block 935, the method 900 may include training the model based on unlabeled data included in an unlabeled dataset.

In some aspects, the training at block 935 may include processing, by the model: reference molecular information included in the unlabeled data; and solvent information of a reference solvent associated with the reference molecular information. The training may include predicting second NMR shifts associated with the reference molecular information based on processing the reference molecular information and the solvent information of the reference solvent.

The training may include comparing the second NMR shifts to observed NMR shifts associated with the reference molecular information. In some aspects, the observed NMR shifts are included in the unlabeled data. The training may include annotating the unlabeled data based on the comparing. The training may include maintaining or updating the model based at least one of the comparing and the annotating.

In some aspects, the training of the model based on the unlabeled data is based on an iterative unsupervised learning strategy which may include iterating between the predicting the second NMR shifts, the comparing the second NMR shifts to observed NMR shifts, and the maintaining or updating the model, in association with satisfying one or more criteria.

In some aspects, the unlabeled dataset is different from an annotated dataset associated with pre-training of the model.

In some aspects, the unlabeled data may include two-dimensional NMR data plotted in a space defined by two frequency axes.

In some embodiments, the training at block 930 and/or the training at block 935 may be implemented before, after, or in parallel to any of the operations described with reference to block 905 through block 925.

In the descriptions of the flowcharts herein, the operations may be performed in a different order than the order shown, or the operations may be performed in different orders or at different times. Certain operations may also be left out of the flowcharts, one or more operations may be repeated, or other operations may be added to the flowcharts.

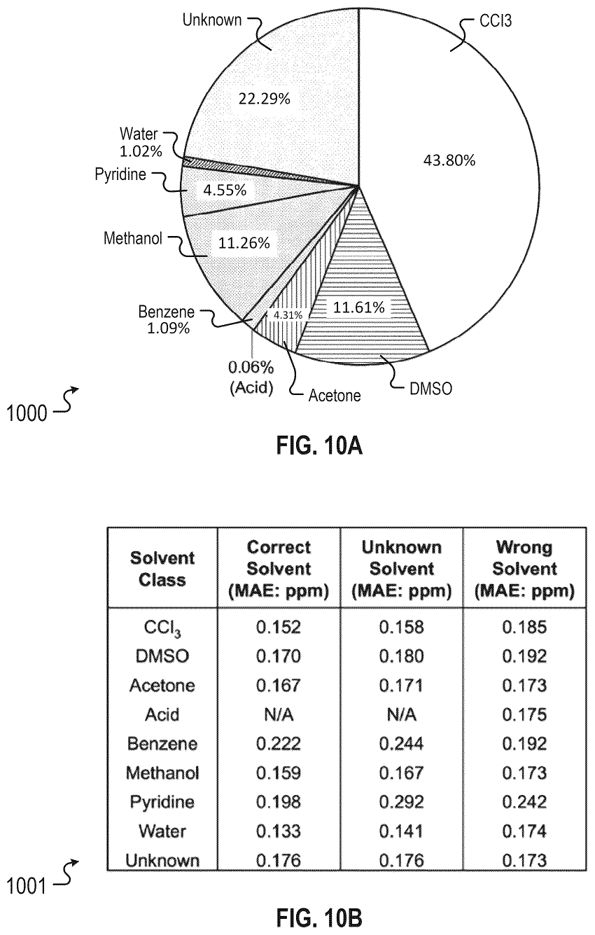

FIG. 10A illustrates a chart 1000 indicating an example solvent distribution of nine solvent classes used in a training dataset for training a model 101 for solvent-aware 2D NMR prediction, in accordance with one or more embodiments of the present disclosure.

FIG. 10B illustrates a table 1001 of solvent effect on proton shift prediction in accordance with one or more embodiments of the present disclosure. As shown at table 1001, when providing the correct solvent information to the model 101, the model 101 provides the most accurate shift prediction compared to cases of unknown solvent information or cases of providing incorrect solvent information. In most cases, specifying the solvent as “unknown” may yield improved performance compared to using an incorrect solvent as input to the model 101. In the example provided in table 1001, the acid solvent environment is marked as “N/A” in the table because it was not captured in the test dataset due to its low presence in the dataset.

Aspects of the present disclosure may take the form of an embodiment that is entirely hardware, an embodiment that is entirely software (including firmware, resident software, micro-code, etc.) or an embodiment combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

The terms “determine,” “calculate,” “compute,” and variations thereof, as used herein, are used interchangeably and include any type of methodology, process, mathematical operation or technique.

While various embodiments of the present disclosure are described herein, it will be understood by those skilled in the art that such embodiments are provided by way of example only. It will be understood by those skilled in the art that numerous modifications and changes to, and variations and equivalent substitutions of, the embodiments described herein can be made without departing from the scope of the disclosure. It is understood that various alternatives to the embodiments described herein may be employed in practicing the disclosure, and modifications may be made to adapt a particular structure or material to the teachings of the disclosure. It is also understood that every embodiment of the disclosure may optionally be combined with any one or more of the other embodiments described herein which are consistent with that embodiment.

Where elements are presented in list format (e.g., in a Markush group), it is understood that each possible subgroup of the elements is also disclosed, and any one or more elements can be removed from the list or group.

It is also understood that, unless clearly indicated to the contrary, in any method described or claimed herein that includes more than one act or step, the order of the acts or steps of the method is not necessarily limited to the order in which the acts or steps of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment found within the prior art, can be explicitly excluded from the claims, regardless of whether or not the specific exclusion is recited in the specification.

Headings are included herein for reference and to aid in locating certain sections. Headings are not intended to limit the scope of the embodiments and concepts described in the sections under those headings, and those embodiments and concepts may have applicability in other sections throughout the entire disclosure.

Unless otherwise defined, all technical and scientific terms used herein have the same meaning as commonly understood by one of ordinary skill in the art to which this invention belongs.

Where a range of values is provided, it is understood that each intervening value between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The term “exemplary” as used herein means “serving as an example, instance or illustration”. Any embodiment or feature characterized herein as “exemplary” is not necessarily to be construed as preferred or advantageous over other embodiments or features.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of’ or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of’ and “consisting essentially of’ shall be closed or semi-closed transitional phrases, respectively.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The term “about” or “approximately” means an acceptable error for a particular value as determined by one of ordinary skill in the art, which depends in part on how the value is measured or determined. In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within 10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” or “less than” applies to each one of the numerical values in that series of numerical values.

All patent literature and all non-patent literature cited herein are incorporated herein by reference in their entirety to the same extent as if each patent literature or non-patent literature were specifically and individually indicated to be incorporated herein by reference in its entirety.

Aspects of the invention is further illustrated by the information and examples set forth in the Appendix (including the References), which are attached hereto after the claims, which is incorporated by reference in its entirety. The examples are non-limiting.

What is claimed is:

1. A method comprising:
processing, by a model:
a molecular graph associated with a molecular structure; and
solvent information of a solvent associated with the molecular structure; and
predicting nuclear magnetic resonance (NMR) shifts associated with the molecular structure based on processing the molecular graph and the solvent information.

2. The method of claim 1, wherein the NMR shifts comprise at least one of:
hydrogen NMR shifts associated with the molecular structure; and
carbon NMR shifts associated with the molecular structure.

3. The method of claim 1, further comprising:
generating a concatenated representation associated with the molecular structure and the solvent, based on processing the molecular graph and the solvent information; and
processing, by one or more multilayer perceptron (MLP) components comprised in the model, the concatenated representation,
wherein predicting the NMR shifts is based on processing the concatenated representation.

4. The method of claim 3, wherein the one or more MLP components comprise:
a first MLP component configured to predict carbon NMR shifts associated with the molecular structure; and
a second MLP component configured to predict hydrogen NMR shifts associated with the molecular structure.

5. The method of claim 1, further comprising training the model based on annotated NMR data comprised in an annotated dataset.

6. The method of claim 5, wherein training the model comprises multi-task pre-training of the model based on the annotated NMR data.

7. The method of claim 5, wherein the annotated NMR data comprises:
annotated one-dimensional hydrogen NMR spectra; and
annotated one-dimensional carbon NMR spectra.

8. The method of claim 5, wherein the annotated NMR data comprises one-dimensional NMR data plotted in a space defined by one frequency axis.

9. The method of claim 1, further comprising training the model based on unlabeled data comprised in an unlabeled dataset, wherein the training comprises:
processing, by the model:
reference molecular information comprised in the unlabeled data; and
solvent information of a reference solvent associated with the reference molecular information;
predicting second NMR shifts associated with the reference molecular information based on processing the reference molecular information and the solvent information of the reference solvent;
comparing the second NMR shifts to observed NMR shifts associated with the reference molecular information, wherein the observed NMR shifts are comprised in the unlabeled data;
annotating the unlabeled data based on the comparing; and
maintaining or updating the model based at least one of the comparing and the annotating.

10. The method of claim 9, wherein the training of the model based on the unlabeled data is based on an iterative unsupervised learning strategy which comprises iterating between the predicting the second NMR shifts, the comparing the second NMR shifts to observed NMR shifts, and the maintaining or updating the model, in association with satisfying one or more criteria.

11. The method of claim 9, wherein the unlabeled dataset is different from an annotated dataset associated with pre-training of the model.

12. The method of claim 9, wherein the unlabeled data comprises two-dimensional NMR data plotted in a space defined by two frequency axes.

13. The method of claim 1, further comprising:
predicting heteronuclear single quantum coherence cross peaks associated with the molecular structure and the solvent based on processing the molecular graph and the solvent information.

14. The method of claim 1, wherein:
the molecular graph is processed in-part by a graph neural network module comprised in the model; and
the solvent information is processed in-part by a solvent encoder comprised in the model.

15. A system comprising:
a processor and a memory, wherein the memory comprises instructions stored thereon that, when executed by the processor, cause the processor to perform operations comprising:
processing, by a model:
a molecular graph associated with a molecular structure; and
solvent information of a solvent associated with the molecular structure; and
predicting nuclear magnetic resonance (NMR) shifts associated with the molecular structure based on processing the molecular graph and the solvent information.

16. The system of claim 15, wherein the NMR shifts comprise at least one of:
hydrogen NMR shifts associated with the molecular structure; and
carbon NMR shifts associated with the molecular structure.

17. The system of claim 15, wherein the instructions, when executed by the processor, further cause the processor to perform operations comprising:
generating a concatenated representation associated with the molecular structure and the solvent, based on processing the molecular graph and the solvent information; and
processing, by one or more multilayer perceptron (MLP) components comprised in the model, the concatenated representation,
wherein predicting the NMR shifts is based on processing the concatenated representation.

18. The system of claim 15, wherein the instructions, when executed by the processor, further cause the processor to perform operations comprising training the model based on one or more of:
annotated NMR data comprised in an annotated dataset; or
unlabeled data comprised in an unlabeled dataset.

19. The system of claim 15, wherein:
the molecular graph is processed in-part by a graph neural network module comprised in the model; and
the solvent information is processed in-part by a solvent encoder comprised in the model.

20. A computer program product comprising a computer readable storage medium having program instructions embodied therewith, the program instructions executable by a processor to cause the processor to perform operations comprising:
processing, by a model:
a molecular graph associated with a molecular structure; and
solvent information of a solvent associated with the molecular structure; and
predicting nuclear magnetic resonance (NMR) shifts associated with the molecular structure based on processing the molecular graph and the solvent information.
